(19) United States
(12) Patent Application Publication
(10) Pub. No.: US 2022/0341263 A1
(43) Pub. Date: Oct. 27, 2022

(54) STATE DETECTION APPARATUS, METHOD AND PROGRAM

(71) Applicant: NIPPON TELEGRAPH AND TELEPHONE CORPORATION, Tokyo (JP)

(72) Inventors: Rie SAKAI, Musashino-shi, Tokyo (JP); Hiroshi YOSHIDA, Musashino-shi, Tokyo (JP); Tomoko SHIBATA, Musashino-shi, Tokyo (JP)

(73) Assignee: NIPPON TELEGRAPH AND TELEPHONE CORPORATION, Tokyo (JP)

(21) Appl. No.: 17/763,734

(22) PCT Filed: Sep. 27, 2019
(86) PCT No.: PCT/JP2019/038279

§ 371 Date: Mar. 25, 2022

(51) Int. Cl. E06C 7/00; G06Q 10/06

(52) U.S. Cl. CPC E06C 7/003; G06Q 10/063114

(57) ABSTRACT

It is possible to easily detect a state of a worker while securing a safety of the worker. A state detection device according to a present embodiment includes a sensor unit, an acquisition unit, a calculation unit, and a determination unit. The sensor unit is disposed on a leg portion of a high-place work tool on which a worker stands. The acquisition unit acquires time-series data related to gravity center sway of the worker from the plurality of sensors. The calculation unit calculates an evaluation value for the gravity center sway from the time-series data. The determination unit determines that a state of the worker working is unstable when the evaluation value is equal to or greater than a threshold value.

### TECHNICAL FIELD

The present invention relates to a state detection device, a method, and a program in a high-place work.

### BACKGROUND ART

Physical injury accidents during high-place work such as telecommunication construction work has become a problem, and, in particular, accidents related to falling of workers have occurred at a constant rate every year.

Thus, a technique of identifying dangerous motions such as swaying and falling of a worker is required. For example, there is a technique for disposing a pressure sensor having a plurality of measurement points on an object on a plane and identifying a motion of a worker from pressure characteristic points when the worker performs a motion on the object on the plane on which the pressure sensor is disposed (see PTL 1, for example). There is also a planar sheet in which a plurality of sensors is arranged in advance (see NPL 1, for example). It is also possible to identify the motion by working by a worker on the planar sheet.

### CITATION LIST

### Patent Literature

PTL 1: JP 2006-223651 A

### Non Patent Literature

NPL 1: Anima Co., Ltd., “BALANCE CODER BW-6000”, [online], [Search on Sep. 9, 2019], internet <URL: https://anima.jp/products/bw6000/>

### SUMMARY OF THE INVENTION

### Technical Problem

Working by a worker on an object or a sheet on which a sensor is disposed is different from working on a scaffold or tread usually used and thus not practical from a viewpoint of safety and cost.

The present invention has been made in view of the above circumstances, and an object of the present invention is to provide a state detection device, a method, and a program capable of easily detecting a state of a worker while securing the safety of the worker.

### Means for Solving the Problem

In order to achieve the above-described object, a state detection device according to one aspect of the invention includes a sensor unit, an acquisition unit, a calculation unit, and a determination unit. The sensor unit is disposed on a leg portion of a high-place work tool on which a worker stands. The acquisition unit acquires time-series data related to gravity center sway of the worker from the plurality of sensors. The calculation unit calculates an evaluation value for the gravity center sway from the time-series data. The determination unit determines that a state of the worker working is unstable when the evaluation value is equal to or greater than a threshold value.

### Effects of the Invention

That is, according to the present invention, it is possible to easily detect a state of a worker while securing a safety of the worker.

### BRIEF DESCRIPTION OF DRAWINGS

FIG. 1 is a block diagram illustrating a state detection system including a state detection device according to a present embodiment.

FIG. 2 is a view illustrating an arrangement example of a sensor unit attached to a high-place work tool.

FIG. 3 is a flowchart illustrating an operation of the state detection device according to the present embodiment.

FIG. 4 is a diagram illustrating an example of management data stored in a work information management database according to the present embodiment.

FIG. 5 is a diagram illustrating an example of age-specific work information stored in the work information management database.

FIG. 6 is a diagram illustrating an example of age-specific work information for each unit time, which is stored in the work information management database.

FIG. 7 is a diagram illustrating an example of a danger detection report output from an output unit according to the present embodiment.

### DESCRIPTION OF EMBODIMENTS

Hereinafter, a state detection device, a method, and a program according to an embodiment of the present disclosure will be described in detail with reference to the drawings. Note that parts with the same numbers applied thereto will not be repeatedly described in the following embodiments on the assumption that the parts perform similar operations.

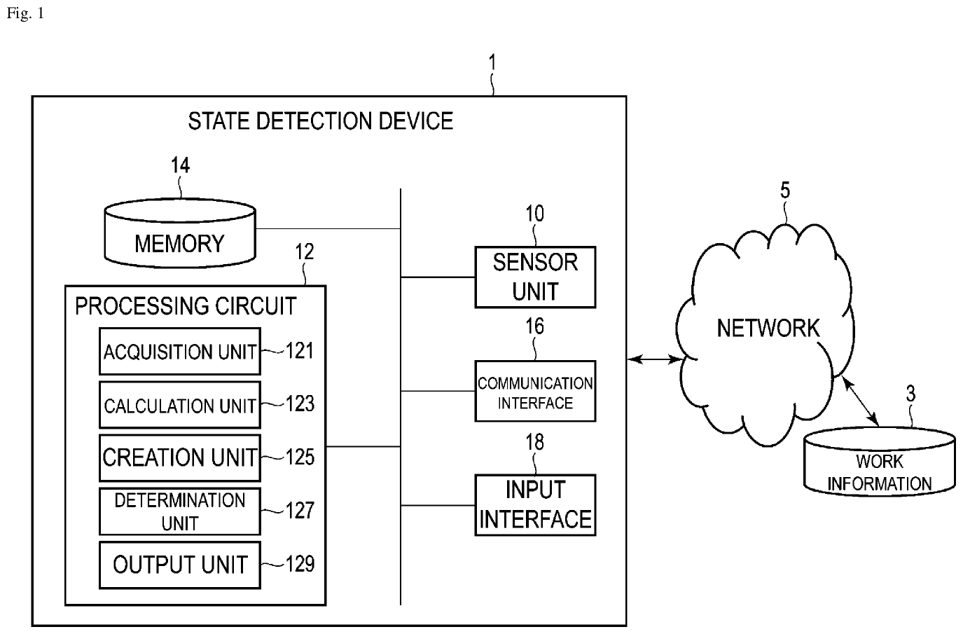

A state detection system including a state detection device according to the present embodiment will be described with reference to FIG. 1.

The state detection system according to the present embodiment includes a state detection device 1 and a work information management database 3.

The state detection device 1 and the work information management database 3 are connected to each other via a network 5 in a wireless or wired manner. Although one state detection device 1 is illustrated in the example illustrated in FIG. 1, a plurality of state detection devices 1 may be connected to one work information management database 3.

The state detection device 1 includes a sensor unit 10, a processing circuit 12, a memory 14, an input interface 18, and a communication interface 16. The processing circuit 12 includes an acquisition unit 121, a calculation unit 123, a creation unit 125, a determination unit 127, and an output unit 129. The sensor unit 10 is connected to other components included in the state detection device 1 in a wired or wireless manner. The processing circuit 12, the memory 14, the communication interface 16, and the input interface 18 are connected to one another, for example, via a bus.

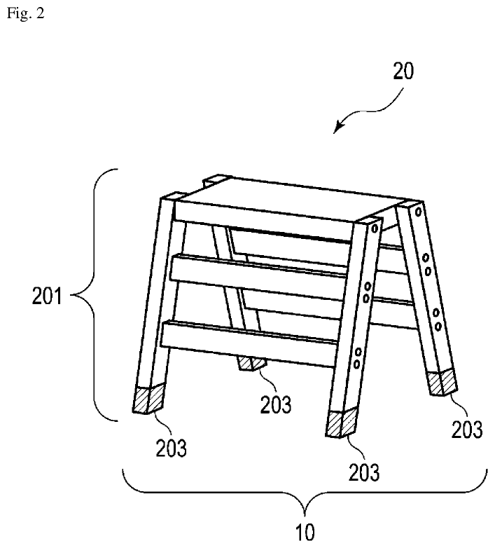

In the sensor unit 10, a plurality of sensors is distributed and disposed at leg portions of a high-place work tool on which a worker stands so that the gravity center of the worker can be calculated. In the present embodiment, description will be made assuming that the high-place work tool is a stepladder, but any tool such as a ladder, a tripod, a work stand, or a scaffold may be used as long as the worker stands on the tool and works at a position higher than the ground. The sensor unit 10 acquires sensor values that change depending on the movement of the center of gravity of the worker. The sensor used as the sensor unit 10 is, for example, a strain sensor capable of measuring a pressure value. The arrangement example of the sensor unit 10 will be described below with reference to FIG. 2.

The acquisition unit 121 acquires time-series data related to the gravity center sway of the worker from the sensor unit 10.

The calculation unit 123 calculates, from the time-series data, an evaluation value for the gravity center sway of the worker. Examples of the evaluation value include a gravity center sway area and a maximum value of variation range of the gravity center trajectory in an individual axial direction.

The creation unit 125 generates work information with reference to the work information management database 3. The work information is data including identification information of a worker, a work start time, a work experience, and the evaluation value. The work experience is, for example, the number of works indicating how many times the work is performed but may be a cumulative work time, and the like.

When the evaluation value is equal to or greater than a threshold value, the determination unit 127 determines that the state of the worker working is unstable. Specific examples of the case where the state of the worker is unstable include a state where the worker is unbalanced and swaying, and a state in which the worker is likely to fall from the high-place work tool.

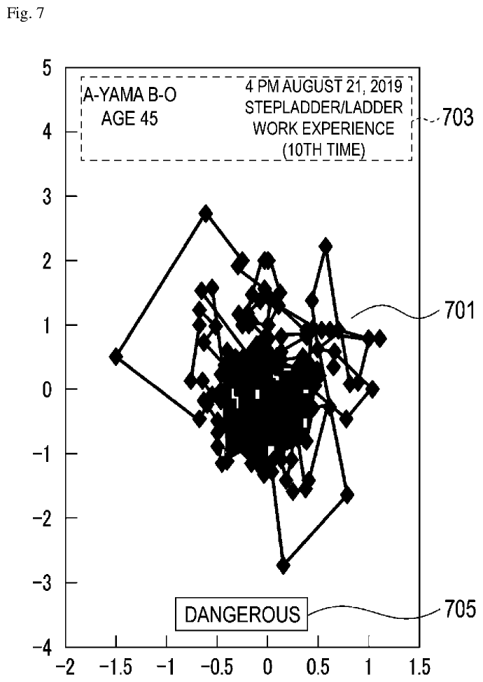

When the determination unit 127 determines that the state of the worker is unstable, the output unit 129 outputs a danger detection report including the identification information of the worker and the evaluation value of the gravity center sway of the worker, based on the work information data. The danger detection report may be transmitted to the work information management database 3 or may be displayed on a display viewable by the worker, another worker, or a manager. The danger detection report will be described below with reference to FIG. 7.

The processing circuit 12 includes a processor such as a central processing unit (CPU) or an integrated circuit such as an application specific integrated circuit (ASIC). Each of the above-described processing units (acquisition unit 121, calculation unit 123, creation unit 125, determination unit 127, and output unit 129) may be implemented as one function of the processor or the integrated circuit by the processor or the integrated circuit executing a processing program.

The memory 14 stores data such as the sensor value, the evaluation value, and the identification information of the worker. For example, the memory 14 may be a generally used storage medium such as a hard disk drive (HDD), a solid state drive (SSD), or a flash memory. In a situation where the state detection device 1 is capable of transmitting and receiving data to and from the work information management database 3 via the network 5, the state detection device 1 may transmit data (sensor value, evaluation value, identification information, and the like) to the work information management database 3 every time the state detection device 1 acquires and generates the data, and thus the memory 14 does not need to store the previous data. In this case, the memory 14 may be a temporary storage medium with a volatile memory such as a cache memory.

The communication interface 16 is an interface for a data communication between the sensor unit 10, the work information management database 3, and the state detection device 1. As the communication interface 16, a commonly used communication interface may be used, and thus descriptions thereof will be omitted.

The input interface 18 is, for example, a mouse, a keyboard, a switch, a button, or a touch panel display and receives an input from a user of the state detection device 1.

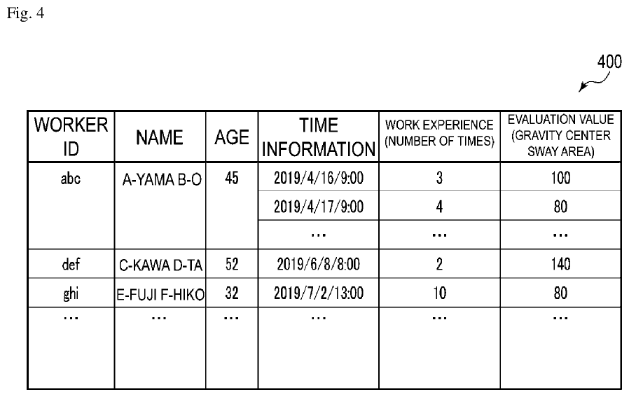

The work information management database 3 stores the work information transmitted from the state detection device 1, the identification information of the worker, the work experience, the threshold value used by the determination unit 127, and the like. The age-specific average work experience and the average evaluation value are also stored based on the stored work information. Although, it is assumed that the work information management database 3 is provided in, for example, a cloud server and communicates with a plurality of state detection devices 1, the work information management database 3 may be stored in a dedicated server. Information stored in the work information management database 3 will be described below with reference to FIGS. 4 to 6.

Next, an example of the sensor unit 10 attached to a stepladder being the high-place work tool on which the worker stands will be described with reference to FIG. 2.

As illustrated in FIG. 2, the sensor unit 10 includes a sensor 203 disposed on each leg 201 of a stepladder 20 on which the worker stands. It is assumed that the sensor 203 is attached to, for example, the tip of the leg 201 of the stepladder 20. A slip resistant grip made of rubber or the like is typically provided on the tip of the leg 201. Thus, the sensor 203 may be disposed between the slip resistant grip and the tip of the leg 201, the sensor 203 may be embedded in the slip resistant grip itself, or a member that has a slip resistant function and includes the sensor unit 10 from the top of the slip resistant grip may be provided at the tip of the leg 201.

It is assumed that the sensor 203 acquires the pressure value as the sensor value, but the sensor may acquire, as the sensor value, other types of information such as the sensing time, the altitude, the temperature, and the magnetic field. In the example of FIG. 2, the respective four sensors 203 may be disposed on the respective legs 201, and thus it is possible to acquire, as the sensor value, the pressure when the worker stands on the stepladder 20 from each of the sensors 203. When the worker stands on the stepladder 20, the pressure on the sensor 203 changes. Thus, it is possible to detect that the worker has stood on the stepladder 20. Furthermore, by continuously acquiring the sensor values from the positions of the four sensors 203 at a predetermined interval, it is possible to calculate the change of the gravity center of the worker from time-series data of the sensor values. The sensor unit 10 only needs to be attached to the tip of the leg of the high-place work tool. Four sensors 203 only need to be provided so long as the high-place work tool is a stepladder. In the case of a ladder, four sensors 203 only need to be provided on the legs which are in contact with the ground and the legs which are in contact with an object to be stood against.

The sensor unit 10 includes a tag recognition unit that senses an ID recognition tag held by the worker. The ID recognition tag includes a worker ID for uniquely identifying the worker. The sensor unit 10 recognizes the ID recognition tag of the worker attempting to stand on the stepladder 20 for a work and acquires the worker ID of the worker standing on the stepladder 20 and the time at which the worker stands on the stepladder. For example, the recognition of the ID tag by the sensor unit 10 may have a configuration capable of recognition in a manner that the worker approaches the ID recognition tag to the sensor unit 10 or brings the ID recognition tag into contact with the sensor unit 10 or have a configuration of enabling the sensor unit 10 to recognize the ID recognition tag which is within a predetermined range from the sensor unit 10. Instead of identifying the worker ID with the ID recognition tag, the worker ID of the worker standing on the stepladder 20 may be identified in a manner that the worker inputs the own worker ID to the input interface 18 of the state detection device 1 and then performs a work.

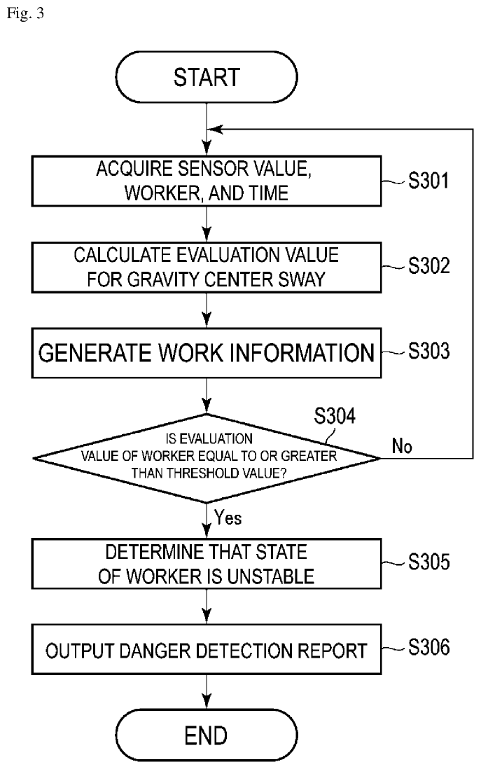

Next, an operation of the state detection device 1 according to the present embodiment will be described with reference to the flowchart in FIG. 3. Here, the stepladder is assumed as the high-place work tool, and a gravity center sway area is assumed as the evaluation value.

In step S301, the acquisition unit 121 acquires, from the sensor unit 10, the sensor value, the worker ID, and the time at which the worker has stood on the stepladder. The time at which the worker has stood on the stepladder is set as the work start time. Specifically, when the sensor unit 10 measures the pressure value and the time as the sensor value, the time included in the sensor value may be acquired as the work start time. When the worker inputs the own worker ID to the input interface 18 of the state detection device 1, and then performs a work, the time at which the worker ID has been input may be set as the work start time.

In step S302, the calculation unit 123 calculates the center of gravity of the worker from the sensor value and calculates the evaluation value for the gravity center sway. Regarding the center of gravity of the worker, if the sensor values at the legs of the stepladder are equal to one another, it can be calculated that the center of gravity of the worker is at the center (for example, center of a work region of the worker, which is defined by the four legs of the stepladder) of a planar region defined by the arrangement of the four sensors. Thus, by comparing the changes of the respective sensor values, it is possible to calculate the position of the center of gravity of the worker in the planar region. In the case of a ladder or the like, it is thought that the sensor value is unbalanced in advance. Alternatively, the values of the sensors before the worker stands on the ladder may be set to an initial state, and the center of gravity of the worker may be calculated by the change in the sensor values. As the gravity center sway area, a general method of calculating an area may be used, for example, an area based on the contour of the trajectory of the center of gravity is used. Thus, description thereof will be omitted. When the evaluation value is the maximum value of the variation range of the gravity center trajectory in axial directions, the maximum value and the minimum value of coordinates of the calculated center of gravity in a vertical direction and a horizontal direction may be calculated, and a difference between the maximum value and the minimum value may be obtained, and then the variation range may be calculated.

In step S303, the creation unit 125 refers to the work information management database 3 to generate work information. Specifically, for example, the creation unit uses the worker ID acquired in step S301 as a key to acquire the name, the age, and the work experience from the work information management database 3, as the identification information of the worker who currently performs a work. The creation unit 125 generates work information including the name, the age, the work start time, the work experience, and the gravity center sway area of the worker. The work experience included in the work information is a value obtained by incrementing a value acquired from the work information management database 3 by one. The creation unit 125 acquires, based on the worker ID, the maximum value (hereinafter, referred to as the maximum gravity center sway area) of the gravity center sway area based on the previous work data of the worker, the average value (hereinafter, referred to as an average gravity center sway area) of the age-specific gravity center sway area, and an average value (hereinafter, referred to as an average work experience) of the age-specific work experience with the high-place work tool.

In step S304, the determination unit 127 determines whether or not the evaluation value of the worker is equal to or greater than a threshold value. Here, the determination unit determines whether or not the gravity center sway area is equal to or greater than the average gravity center sway area at the age of the worker. When the evaluation value is equal to or greater than the threshold value, that is, the gravity center sway area is equal to or greater than the average gravity center sway area, the process proceeds to step S305. When the evaluation value is smaller than the threshold value, that is, the gravity center sway area is smaller than the average gravity center sway area, the process returns to step S301 and the similar processes are repeated.

That is, the sensor value is time-series data sampled at a predetermined interval. Thus, by repeating the processes of steps S301 and S302, it is possible to calculate the trajectory of the center of gravity every time a new sensor value is acquired as the time-series data. Thus, it is possible to calculate the gravity center sway area in real time. The process of acquiring the identification information of the worker from the work information management database 3 in step S303 only needs to be performed once. Thus, in the repetition, the process of acquiring the identification information of the worker may be omitted.

In step S304, the determination unit 127 does not perform comparison with the maximum gravity center sway area but may determine whether or not the gravity center sway area is equal to or greater than the maximum gravity center sway area of the worker and the work experience of the worker is equal to or greater than the age-specific average work experience. When the gravity center sway area is equal to or greater than the maximum gravity center sway area of the worker and the work experience of the worker is equal to or greater than the average work experience, the process proceeds to step S305. When gravity center sway area is smaller than the maximum gravity center sway area, or the work experience is smaller than the average work experience, the process returns to step S301 and the similar processes are repeated. The reason that, when the work experience is smaller than the average work experience, the worker is not determined to be unstable is as follows. That is, it is thought that, because the worker is not accustomed to the stepladder work itself, accurate information related to the gravity center sway is not obtained.

In step S305, the determination unit 127 determines that the state of the worker working is unstable.

In step S306, the output unit 129 outputs a danger detection report including a graph of the gravity center sway area determined to be unstable, based on the work information created in step S303.

Next, FIG. 4 illustrates an example of management data stored in the work information management database 3.

As the management data, the worker ID, the name, the age, time information, the work experience, and the evaluation value are stored in a management data table 400 in association with each other.

The time information is the work start time of the worker. A time at which the worker gets off the high-place work tool may be set as a work end time, the work time may be calculated from a difference between the work end time and the work start time, and the work time may be stored as the time information.

It is assumed that the work experience is the number of times indicating how many times the work is performed, in the present embodiment. The work experience may be a cumulative work time or the number of years of experience, and only needs to be a value representing the experience related to the work of the worker.

When the work information transmitted from the state detection device 1 is received, and the work start time included in the work information is different from the work start time of the same worker ID, which is already stored in the management data table 400, items of the time information, the work experience, and the evaluation value are newly added to the work information management database 3 illustrated in FIG. 4, as entries for the same worker ID. At this time, regarding the work experience, a value obtained by incrementing one from the value of the work experience which is already stored is stored.

In the example of FIG. 4, regarding a person with the worker ID “abc”, the name “A-YAMA B-O”, and the age “45”, entries of time information “2019/4/16/9:00”, a work experience (number of times) “3”, and an evaluation value (gravity center sway area) “100” and entries of time information “2019/4/17/9:00”, a work experience (number of times) “4”, and an evaluation value (gravity center sway area) “80” are stored in association with each other. Thus, the creation unit 125 can obtain the identification information of a worker with the name “A-YAMA B-O”, the age “45”, and the work experience (number of times) “3” by using the worker ID “abc” as the key.

The latest work data may be stored for the worker ID without leaving a history of the previous work experience. That is, in the example of FIG. 4, only the entry for the work experience “4” may be stored. At this time, the previous time information, the work experience, and the gravity center sway area may be stored as separate items in association with the worker ID.

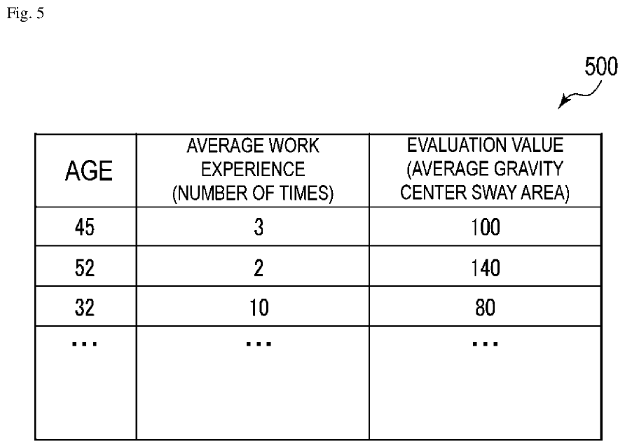

Next, an example of the age-specific work information stored in the work information management database 3 will be described with reference to FIG. 5.

In FIG. 5, the age-specific work information is stored in an age-specific work information table 500 in association with the age, the average work experience, and the evaluation value (average gravity center sway area).

The age is not limited to the age such as the age of 20 or the age of 32, and an age layer having a range of “30 years to 35 years” may be used. Regarding the age-specific average work experience and the evaluation value (average gravity center sway area), the average work experience and the average gravity center sway area may be calculated by accumulating pieces of the work data (as illustrated in FIG. 4) obtained from a plurality of state detection devices 1, and performing an analysis such as taking an average by age.

With fatigue due to a work for a long time, the shape of the gravity center sway area may be changed. Thus, in this case, the average gravity center sway area or the maximum gravity center sway area of the worker is stored for each unit time. The determination unit 127 may change the average gravity center sway area or the maximum gravity center sway area of the worker to the average gravity center sway area or the maximum gravity center sway area of the worker for the corresponding unit time, in accordance with the length of the work time of the worker, and determine whether or not the state of the worker is unstable.

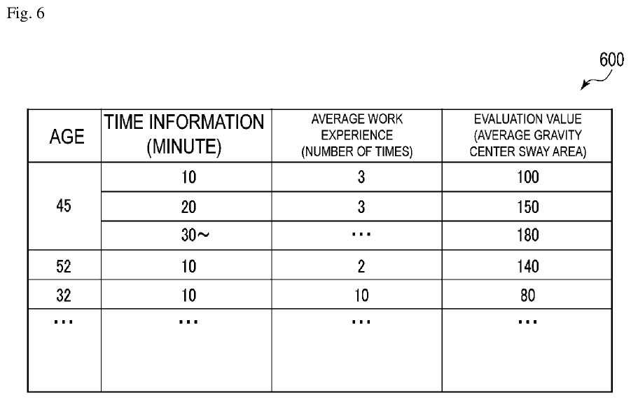

An example of the age-specific work information for each unit time, which is stored in the work information management database 3, will be described with reference to FIG. 6.

An age-specific work information table 600 illustrated in FIG. 6 is different from the age-specific work information table 500 illustrated in FIG. 5 in that the age-specific work information table 600 includes entries of the time information and the evaluation value (average gravity center sway area) for each unit time. Here, 10 minutes are assumed as the unit time.

For example, in a work from the start to 10 minutes, the determination unit 127 may determine the state of the worker by using the average gravity center sway area “100” as a reference and determining whether or not the currently-measured gravity center sway area is equal to or greater than “100”.

Then, in a work from 10 minutes to 20 minutes as the next unit time, the center of gravity of the worker may be slightly shifted by fatigue, and thus the average gravity center sway area is slightly increased. The determination unit 127 may determine the state of the worker by using the average gravity center sway area “150” as the reference and determining whether or not the currently-measured gravity center sway area is equal to or greater than “150”. Thus, it is possible to improve the detection accuracy of unstableness of the state of the worker.

Regardless of the work time, it is necessary to determine a value which is set as the upper limit of the average gravity center sway area, and is used for determining the danger, for example, when the gravity center sway area is equal to or greater than this value. Thus, when the work time is equal to or longer than a predetermined time, the average gravity center sway area is set to a predetermined value regardless of the unit time. For example, when the work time is equal to or longer than 30 minutes, the average gravity center sway area may be set to “180”. Thus, in a case of being equal to or longer than a predetermined work time, the determination unit 127 may determine the state of the worker by determining whether or not the currently-measured gravity center tuning area is equal to or greater than “180”.

Next, FIG. 7 illustrates an example of the danger detection report output from the output unit 129.

FIG. 7 illustrates an example in which a graph 701 for the trajectory of the gravity center sway is displayed as the danger detection report, and work data 703 and a message 705 are superimposed and displayed on the graph 701. The message may be an expression indicating that the state of the worker is unstable and dangerous. Specifically, as the work data, the name “A-YAMA B-O”, the age “45”, the start time “4 pm, Aug. 21, 2019”, and the work experience “stepladder/ladder experience (10th time)” are displayed on the top of the graph 701. The message 705 “dangerous” is displayed at the bottom of the graph 701.

When the worker views the danger detection report illustrated in FIG. 7, it is possible to objectively grasp the unstableness that cannot be perceived by the sense of the worker. In addition, when another worker or the manager views the danger detection report, it is possible to grasp a sign such as swaying more than usual, and to perform danger prediction of grasping a dangerous sign in advance.

According to the present embodiment described above, the sensor is attached to the leg portion of a high-place work tool such as a ladder and a stepladder, and the danger detection report is output when it is determined that the worker is unstable, by using the evaluation value such as the gravity center sway area. Thus, the worker can determine the unstableness such as the danger of swaying or falling of the worker only by continuing the work while standing on the high-place work tool used by the worker, without performing a useless work that the worker stands on a reaction plate or the like separately from the original work and measures the gravity center sway. In addition, by outputting the danger detection report, it is possible to visualize a dangerous sign, and to cause the sign to be known to the worker or the surroundings. As a result, it is possible to easily detect the state of the worker while securing the safety of the worker.

The instructions indicated in the processing procedures described in the embodiment described above can be executed by a computer based on a software program.

In short, the present invention is not limited to the above-described embodiment as it is and can be embodied with the components modified without departing from the scope of the disclosure when implemented. Furthermore, various inventions can be formed by appropriate combinations of a plurality of components disclosed in the above-described embodiment. For example, several components may be deleted from all of the components illustrated in the embodiments. Furthermore, components of different embodiments may be appropriately combined with each other.

### REFERENCE SIGNS LIST

1 State detection device
3 Work information management database
5 Network
10 Sensor unit
12 Processing circuit
14 Memory
16 Communication interface
18 Input interface
20 Stepladder
30 Work time
121 Acquisition unit
123 Calculation unit
125 Creation unit
127 Determination unit
129 Output unit
201 Leg
203 Sensor
400 Management data table
500, 600 Age-specific work information table
701 Graph
703 Work data
705 Message

1. A state detection device comprising:
a sensor unit disposed on a leg portion of a high-place work tool on which a worker stands;
a processor; and
a storage medium having computer program instructions stored thereon, when executed by the processor, perform to:
acquire time-series data related to gravity center sway of the worker from the sensor unit;
calculate an evaluation value for the gravity center sway from the time-series data; and
determine that a state of the worker working is unstable when the evaluation value is equal to or greater than a threshold value.

2. The state detection device according to claim 1, wherein the computer program instructions further perform to calculates, as the evaluation value, a value including a gravity center sway area of the worker or a maximum value of a variation range in an individual axial direction of a gravity center trajectory of the worker.

3. The state detection device according to claim 1, wherein
the evaluation value is a gravity center sway area, and
the computer program instructions further perform to determines that the state of the worker is unstable when the gravity center sway area of the worker is equal to or greater than an average value of a gravity center sway area corresponding to an age of the worker, or when the gravity center sway area is equal to or greater than a maximum gravity center sway area in a previous work of the worker and a work experience of the worker is equal to or greater than an average value of a work experience corresponding to the age of the worker.

4. The state detection device according to claim 3, wherein the computer program instructions further perform to changes the average value of the gravity center sway area and the maximum gravity center sway area depending on a length of a work time of the worker and determines whether the state of the worker is unstable.

5. The state detection device according to claim 1, further comprising:
an output unit configured to output a danger detection report including identification information of the worker and the evaluation value of the worker when it is determined that the state of the worker is unstable.

6. A state detection method comprising:
acquiring time-series data related to gravity center sway of a worker from a sensor unit disposed on a leg portion of a high-place work tool on which the worker stands;
calculating an evaluation value for the gravity center sway from the time-series data; and
determining that a state of the worker working is unstable when the evaluation value is equal to or greater than a threshold value.

7. A program for causing a computer to operate as an individual unit of the state detection device described in claim 1.
